(19) United States
(12) Patent Application Publication
(10) Pub. No.: US 2026/0069547 A1
(43) Pub. Date: Mar. 12, 2026

(54) MICROCAPSULES, PROCESS FOR THE PREPARATION OF MICROCAPSULES AND USE OF MICROCAPSULES FOR PERFUMING A CONSUMER PRODUCT

(71) Applicant: SYMRISE AG, Holzminden (DE)

(72) Inventors: Amadeo CABRERA, Mexico City (MX); Alejandro CASTRO, Mexico City (MX); John TEFFENHART, Edison, NJ (US)

(21) Appl. No.: 19/124,520

(22) PCT Filed: Nov. 30, 2022
(86) PCT No.: PCT/EP2022/083843

§ 371 Date: Apr. 25, 2025

Related U.S. Application Data
(60) Provisional application No. 63/382,133, filed on Nov. 3, 2022.

(51) Int. Cl. A61K 9/50; A61K 8/11; A61Q 13/00; C11D 17/00

(52) U.S. Cl. CPC A61K 9/5031; A61K 8/11; A61Q 13/00; C11D 17/0039

(57) ABSTRACT

The present invention relates to the field of perfumed particles in particular for solid consumer product formulations such as textile care products or the like. More specifically, the present invention relates to microcapsules and a process for the preparation of microcapsules. Moreover, the present invention relates to microcapsules obtained by the process. The present invention further relates to a consumer product comprising at least one microcapsule according to the invention and the use of microcapsules for perfuming a consumer product according to the invention.

### FIELD OF THE INVENTION

The present invention relates to the field of perfumed particles in particular for solid consumer product formulations such as textile care products or the like. More specifically, the present invention relates to microcapsules and a process for the preparation of microcapsules. Moreover, the present invention relates to microcapsules obtained by the process. The present invention further relates to a consumer product comprising at least one microcapsule according to the invention and the use of microcapsules for perfuming a consumer product according to the invention.

### BACKGROUND OF THE INVENTION

Nowadays many consumer products such as detergents, fabric softeners, washing powders, deodorants, lotions, etc. are perfumed with fragrance materials or contain cosmetic ingredients to deliver a specific effect.

WO 2008/098 387 A1 discloses microcapsules comprising a core of fragrance and a shell of an aminoplast polymer, wherein the shell comprises a thermoset resin, a terpolymer and a polymeric stabilizer. The terpolymer further comprises moieties derived from at least one polyamine, moieties derived from at least one aromatic polyol and moieties having methylene units.

EP 3 099 404 B1 discloses one-shell aminoplast core-shell microcapsules crosslinked with a polyisocyanate and encapsulating a perfume oil and provides use of said microcapsules in liquid aqueous and powder surfactant-rich consumer products.

WO 2020/114 975 A1 describes microcapsules comprising a core, containing at least one active ingredient, and a capsule wall, the capsule wall consisting of at least three polymer layers and at least one of the polymer layers consisting of a first phenol resin, the first phenol resin comprising groups that derive from at least one aromatic polyol, and at least one further of the polymer layers consisting of a second phenol resin, the second phenol resin comprising groups that derive from at least one triphenol.

WO 2016/028 875 A1 discloses methods of preparing a capsule delivery system having two or more different capsules. Further disclosed are capsule delivery systems and consumer products containing such a capsule delivery system.

US 2014 322 283 A1 discloses to water-dispersible core-shell microcapsules essentially free of formaldehyde. In particular it concerns core-shell microcapsules having a shell obtained by reacting polyisocyanate or polyoxirane cross-linkers and oligomeric compositions which are the reaction products between a polyamine component and a particular mixture of glyoxal and a 2,2-dialkoxy-ethanal.

US 2014/378 367 A1 discloses one-shell aminoplast core-shell microcapsules stabilized by a polyisocyanate and further describes a method for stabilizing aminoplast microcapsules in liquid aqueous surfactant-rich consumer products.

US 2016 354 749 A1 describes a process for the preparation of organic-inorganic microcapsules, with a flavour or fragrance core and a hybrid shell composed of at least two types of inorganic particles that are cross-linked.

WO 2012/107 323 A1 relates to polyurea core-shell microcapsules having a polyurea shell comprising the reaction product of a polyisocyanate with guanazole and an amino acid.

U.S. Pat. No. 9,271,905 B2 discloses a process for producing perfume-containing microcapsules with a polyurea wall that can be used in home or personal care products, as well as to the microcapsules themselves and consumer products that contain these microcapsules. The process of the invention uses a combination of aromatic and aliphatic polyisocyanates in specific relative concentrations.

Unfortunately, often, for example, the fragrant substances or cosmetic ingredients of such products interact with other components of the product formulation, or the more volatile components of the perfume formulation tend to evaporate prematurely. Consequently, this leads to an undesired change and/or decrease of the fragrance impression or premature “use” of the cosmetic ingredient over time or might even cause disadvantageous reactions with the other ingredients of the product formulation resulting in a reduced product quality and/or stability. In order to prevent possible interactions of the fragrances or other active components with the other ingredients of the product or, for example, to prevent the volatilization of the fragrances and thus not to distort or reduce the desired olfactory impression, the fragrances or other active ingredients can be added to the formulation in encapsulated form. In this way, for example the desired olfactory impression can be guaranteed based on a targeted release of the active component. Moreover, interactions between the product components can be reduced allowing for improved product quality and storage stability.

Therefore, a broad variety of consumer products comprise encapsulated active ingredients such as fragrances in their formulations. For example, textile care formulations such as liquid detergents or fabric softener comprise a plurality of such perfumed microcapsules. However, the incorporation of microcapsules into powdered formulations is considerably aggravated as a mixing of the microcapsule component and the other detergent components leads to breakage of the fragile microcapsules resulting in a loss of for example olfactory performance. It is particularly difficult to produce microcapsules that have both good stability and good release properties. The ability of the capsules to retain the active ingredient and thus to prevent the loss of volatile components depends in particular on the stability of the capsules in the product base. However, very stable microcapsules, e.g. capsules having a comparably thick capsule wall, usually tend to exhibit a low performance as the break of the microcapsules and thus the release of the active ingredient(s) is hindered. However, if they are too unstable, they are already destroyed during storage or result in a leakage of the active ingredient and do not perform either. Thus, an increase of the capsule wall, i.e. multi-layered microcapsules shell or a thicker shell, respectively, likewise result in a decreased performance as the targeted release of the active ingredient is aggravated and said microcapsules usually require a complex multi-step preparation process.

In addition, there is a new and increasing trend towards solid consumer products such as solid hair care products, soaps and solid body wash in view of the public's increased ecological awareness (less waste, less preservatives, etc.), so that solid consumer products will become more and more relevant not only in the cosmetic industry but also in other industries.

Consequently, there is a need to improve the efficient incorporation of actives such as perfuming ingredients into solid consumer product formulations, which however, simultaneously allow for an efficient and targeted release of said active.

Particularly, there is a need to provide said formulations comprising said actives, wherein said actives are provided with improved stabilities and improved release properties.

### SUMMARY OF THE INVENTION

The present invention was made in view of the disadvantages described above. To solve the above-mentioned drawbacks, the present invention provides microcapsules, a method for the preparation thereof, allowing for the efficient incorporation of active ingredients with improved stabilities into solid consumer product formulations for the preparation of consumer products further providing improved release properties.

Thus, in a first aspect the present invention relates to microcapsules, comprising at least one ingredient, preferably comprising at least one fragrance, and at least three different layers surrounding said ingredient, wherein at least one layer is a polyurethane layer and at least one layer is one of a melamine-formaldehyde layer or a phenolic resin layer.

In a preferred embodiment, the microcapsules comprise at least one polyurethane layer and at least one melamine-formaldehyde layer and at least one phenolic resin layer.

More preferably, the at least one melamine-formaldehyde layer is arranged between the at least one polyurea layer and the at least one phenolic resin layer.

Preferably, the at least three different layers are separated.

In a second aspect the present invention relates to a process for the preparation of microcapsules containing at least one ingredient, preferably comprising at least one fragrance, the microcapsules comprise at least three layers surrounding said ingredient, wherein in a first step (a) at least a first layer is prepared, wherein in a second step (b), at least a second layer is prepared and wherein in a third step (c), at least a third layer is prepared, wherein the second layer is prepared after the first layer is hardened and the third layer is prepared after the second layer is hardened.

According to a third aspect the present invention relates to microcapsules obtained by the process according to present invention.

Additionally, in a further aspect of the present invention relates to a consumer product selected from the group consisting of detergents and cleaning agents, personal care products, nutritional or recreational preparations, cosmetic or pharmaceutical preparations, perfumed, perfumed or products to be perfumed, preferably fragrance rinsers, laundry perfumes, the product comprising at least one microcapsule according to the invention.

Finally, the present invention relates to the use of microcapsules according to the invention for perfuming a consumer product, preferably a consumer product according to the present invention.

That is, the inventors have found that the microcapsules according to the invention allow an efficient incorporation of active ingredients in encapsulated form which show excellent release properties (performance) into a broad range of solid consumer product formulations and consumer products as such. Thereby, the at least three layers are protecting the fragile microcapsules during the preparation of the final consumer product (formulation) from mechanical, chemical and/or thermal influences without negatively influencing the performance of the microcapsules within the consumer product (formulation). Based thereon the present dosage form allows for an efficient incorporation of active ingredients via microcapsules in solid consumer product formulations while simultaneously allowing for an efficient and targeted release of said active ingredients thus overcoming the drawbacks of the state of the art.

The present problem is solved by the objects of the independent patent claims. Preferred embodiments are apparent from the wording of the dependent patent claims as well as the following description.

All percentages are by weight unless otherwise indicated and based on the total weight of the microcapsule. Numeric examples given in the form “x to y” include the values given. When multiple preferred numeric ranges are specified in this format, all ranges created by combining the various endpoints are also included.

The terms “at least one” or “one or more” as used herein refers to 1 or more, for example 2, 3, 4, 5, 6, 7, 8, 9 or more.

The term “and/or” expresses that a linkage exists, or an alternative is provided.

### BRIEF DESCRIPTION OF DRAWINGS

FIG. 1: FIG. 1 shows the structure of microcapsules according to present invention schematically;

FIG. 2: FIG. 2 shows sensory evaluation results of the performance (i.e. release properties) of microcapsules according to the invention.

### DETAILED DESCRIPTION OF THE INVENTION

Hereinafter, the present invention is described in more detail.

Thus, in a first aspect the present invention relates to microcapsules, comprising at least one ingredient, preferably comprising at least one fragrance, and at least three different layers surrounding said ingredient, wherein at least one layer is a polyurethane layer and at least one layer is one of a melamine-formaldehyde layer or a phenolic resin layer. It was surprisingly found that such microcapsules have improved stability properties next to excellent release properties.

In a further preferred embodiment microcapsules with yet improved stability properties comprise at least one polyurethane layer and at least one melamine-formaldehyde layer and at least one phenolic resin layer.

Preferably, in a further preferred embodiment the at least one melamine-formaldehyde layer is arranged between the at least one polyurea layer and the at least one phenolic resin layer. This could lead to simplified preparation of the microcapsules.

Preferably, in a yet further embodiment the at least one phenolic resin layer is an outer layer of the microcapsules. This could lead to simplified preparation of the microcapsules and further, this could enable to improve the stability of the microcapsules.

It was surprisingly found that microcapsules with excellent release properties (performance) could be obtained wherein the at least one polyurethane layer is formed by tannic acid and at least one polyisocyanate, preferably at least one aromatic polyisocyanate, the at least one melamine-formaldehyde layer is formed by at least one melamine-formaldehyde precondensate in water or by at least melamine and formaldehyde as single compounds and the at least one phenolic resin layer is formed by at least one aromatic polyol, preferably by Phloroglucinol and/or Resorcinol.

Preferably, the at least one aromatic polyisocyanate is at least a difunctional, preferably a polyfunctional polyisocyanate. In preferred embodiments, the at least one aromatic polyisocyanate contains an average of 2 to 5 —N═C═O functional groups. These include, for example, aromatic di-, tri-and higher polyisocyanates.

Preferably, the at least one polyisocyanate does not contain less than 2 —N═C═O functional groups. Particularly, the at least one aromatic polyisocyanate is not a monoisocyanate.

According to the invention, the at least one polyisocyanate can be a mixture comprising two or more different polyisocyanates, preferably aromatic polyisocyanates each comprising two or more —N═C═O functional groups.

In a preferred embodiment, the at least one polyisocyanate is a mixture of an aromatic polyisocyanate comprising 2 —N═C═O functional groups (diisocyanate) and an aromatic polyisocyanate comprising 3 —N═C═O functional groups (triisocyanate), wherein the at least on polyisocyanate with 3 —N═C═O functional groups (triisocyanate) is obtained from said aromatic polyisocyanate comprising 2 —N═C═O functional groups (diisocyanate).

It was found that capsules with excellent release properties were obtained by using a mixture of an aromatic diisocyanate and an aromatic polyisocyanate comprising 3 or more —N═C═O functional groups. Preferably, the amount of aromatic diisocyanate in a mixture comprising an aromatic diisocyanate and an aromatic polyisocyanate comprising 3 or more —N═C═O functional groups is 0.2 wt. % to 0.6 wt. %, more preferably 0.25 wt. % to 0.4 wt. % and particularly 0.3 wt. %.

In a further embodiment of the invention, the at least one polyisocyanate is a mixture of an aromatic diisocyanate and an aromatic triisocyanate, wherein the aromatic diisocyanate comprises or consist of xylylene diisocyanate, more preferably m-xylylene diisocyanate.

Capsules with excellent release properties could be obtained, if the at least one polyisocyanate is a mixture of an aromatic diisocyanate and an aromatic triisocyanate, wherein the aromatic diisocyanate comprises or consist of xylylene diisocyanate, more preferably m-xylylene diisocyanate, wherein the amount of the aromatic diisocyanate is 0.2 wt. % to 0.6 wt. %, more preferably 0.25 wt. % to 0.4 wt. % and particularly 0.3 wt. %.

In a preferred embodiment, the at least one polyisocyanate is a mixture of an aromatic polyisocyanate comprising 2 —N═C═O functional groups (diisocyanate) and an aromatic polyisocyanate comprising 3 —N═C═O functional groups (triisocyanate), wherein the at least on polyisocyanate with 3 —N═C═O functional groups (triisocyanate) is obtained from said aromatic polyisocyanate comprising 2 —N═C═O functional groups (diisocyanate).

Moreover, it was surprisingly found that microcapsules according to present invention, wherein the at least three different layers are separated layers show excellent release properties (performance).

Such a separation can be realized particularly by building up the microcapsules layer-by-layer with separate hardening steps after each layer-formation. The layers thus obtained are distinct, preferably non-linked.

In general, microcapsules are particles comprising a core and a wall material surrounding the core, wherein the core may be a solid, liquid or gaseous substance or substance mixture surrounded by a polymeric dense, permeable or semi-permeable wall material. The wall is usually formed by precipitation of the polymers upon emulsification and coacervation or interfacial polymerization. The core is also referred to as the inner phase. Other names used for the wall include outer phase, shell or coating. The shell wall can comprise one or more layers and can comprise an additional coating with a coating substance. The diameter of the microcapsules typically varies in the range of 1 to 1000 μm. The wall thickness is typically 0.5 to 150 μm but can be varied in the range of 5·10⁻⁹ m to 5·10⁻⁶ m. Typically loadings of 25 to 95 wt. % with core material, but also those of 1 to 99 wt. % are possible.

For example, hydrophobic active ingredients, such as fragrances or aroma substances, can be easily incorporated into numerous and diverse application formulations through encapsulation.

Based on their properties, microcapsules are used in the printing industry, food industry (vitamins, flavours, plant extracts, enzymes, microorganisms), agricultural chemistry (fertilizers, pesticides), feed industry (minerals, vitamins, enzymes, drugs, microorganisms), pharmaceutical industry, detergent industry, and cosmetic industry, among others.

A variety of capsule wall or coating materials are known for the manufacture of microcapsules. The capsule wall can be made of either natural, semi-synthetic or synthetic materials. Natural shell materials include gum arabic, agar-agar, agarose, maltodextrins, alginic acid or its salts, e.g., sodium alginate or calcium alginate, fats and fatty acids, cetyl alcohol, collagen, chitosan, lecithins, gelatine, albumin, shellac, polysaccharides such as starch or dextran, polypeptides, protein hydrolysates, sucrose, and waxes. Semisynthetic capsule wall materials include chemically modified celluloses, especially cellulose esters, and cellulose ethers, e.g., cellulose acetate, ethyl cellulose, hydroxypropyl cellulose, hydroxypropyl methyl cellulose, and carboxymethyl cellulose, as well as starch derivatives, in particular starch ethers and starch esters. Synthetic shell materials are, for example, polymers such as polyacrylates, polyamides, polyvinyl alcohol or polyvinylpyrrolidone.

Depending on the type of capsule wall material and manufacturing process, microcapsules with different properties in terms of diameter, size distribution, and physical and/or chemical properties are formed in each case.

Preferably, the microcapsules used within the scope of the present invention are such microcapsules, wherein the shell of the at least one microcapsule comprises or consists of a shell material selected from the group consisting of: sol-gel polymer (e.g., silica), polysiloxane, polyacrylate, polyacrylamide, poly(acrylate-co-acrylamide), polyurea, polyurethane, polyamide, polypeptide, polysulfonate, polysaccharide, polyphenolic polymers, poly(melamine-formaldehyde), poly(resorcinol-formaldehyde), poly(urea-formaldehyde), poly(melamine-urea), or combinations thereof.

Preferably, the microcapsules according to the present invention are selected from the following group of capsule types consisting of: poly(melamine-formaldehyde)-based microcapsules and isocyanate-based microcapsules such as polyurea-based and/or polyurethane-based microcapsules which are preferably biodegradable.

In the context of the present invention, microcapsules are understood to be microparticles comprising at least one or more active ingredient(s) as core material inside the capsule and which are enclosed by a capsule shell or capsule wall as specified above. As active ingredient to be encapsulated according to the invention, basically any material suitable for inclusion in microcapsules can be considered in the process according to the invention. Preferably, hydrophobic or lipophilic, i.e., water-insoluble, or water-immiscible liquids or solids as well as suspensions can be considered as active ingredients to be encapsulated. These are predominantly non-polar substances. Such hydrophobic substances are almost always lipophilic, i.e., they dissolve well in fat and oil. The terms “microcapsule(s)” and “capsule” or “hydrophobic” and “lipophilic”are used synonymously within the context of the present invention.

In the context of the present description, the core material is preferably a hydrophobic active substance, i.e., a substance that has a specific effect or causes a specific reaction, for example, a drug, a pesticide, a cosmetic active ingredient, a food active ingredient, etc. Particularly, the hydrophobic active substance is a lipophilic or hydrophobic fragrance or aroma substance or a lipophilic or hydrophobic perfume oil or aroma (i.e. a mixture of fragrance substances or aroma substances, respectively), a cooling agent, a TRPV1 or a TRPV3 modulator, a substance that produces a pungent taste or a warmth or heat sensation on the skin or mucous membranes or a tingling sensation in the mouth or throat, or active ingredients with astringent effect, a pesticide, a biocide, an insecticide, a substance from the group of repellents, a food additive, a cosmetic active ingredient, a pharmaceutical active ingredient, a dye, a dye precursor; an agrochemical, a dye, a luminous paint, an optical brightener, a solvent, a wax, a silicone oil, a lubricant, a print coating for paper, or a mixture of two or more of the aforementioned active ingredients.

The microcapsules in the context of present invention might have a core material in the form of a hydrophobic single fragrant substance or single odorant substance, wherein the core material comprises at least one single fragrant substance or single odorant substance or mixtures thereof, selected from one or more of the following groups:
Extracts of natural raw materials and also fractions thereof or components isolated therefrom; single fragrance substances from a group of hydrocarbons; aliphatic alcohols; aliphatic aldehydes and acetals; aliphatic ketones and oximes; aliphatic sulfur-containing compounds; aliphatic nitriles; esters of aliphatic carboxylic acids; formates, acetates, propionates, isobutyrates, butyrates, isovalerates, pentanoates, hexanoates, crotonates, tiglinates and 3-methyl-2-butenoates of acyclic terpene alcohols; acyclic terpene aldehydes and ketones as well as their dimethyl and diethyl acetals; formates, acetates, propionates, isobutyrates, butyrates, isovalerates, pentanoates, hexanoates, crotonates, tiglinates and 3-methyl-2-butenoates of cyclic terpene alcohols; cyclic terpene aldehydes and ketones; cyclic alcohols; cyclic and cycloaliphatic ethers; cyclic and macrocyclic ketones; cycloaliphatic aldehydes; cycloaliphatic ketones; esters of cyclic alcohols; esters of cycloaliphatic carboxylic acids; aromatic hydrocarbons; araliphatic alcohols; esters of araliphatic alcohols and aliphatic carboxylic acids; araliphatic ethers; aromatic and araliphatic aldehydes; aromatic and araliphatic ketones; aromatic and araliphatic carboxylic acids and their esters; nitrogen-containing aromatic compounds; phenyl ethers and phenyl esters; heterocyclic compounds; lactones; and mixtures of the above active ingredients.

Specific examples of the abovementioned substances are known to the person skilled in the art. Fragrant substances or odorant substances and mixtures of two or more of said substances are chemical substances or compositions which are used to confer, impart and/or modulate a specific odour or odour impression. Preferably, said odour (impression) is considered as being pleasant.

In an alternative embodiment according to the present invention, the microcapsules according to the invention use a fragrance substance or a perfume oil, respectively, or an aroma substance or an aroma as the active ingredient to be encapsulated or as the core material. These are compositions containing at least one fragrance substance or one aroma substance. Such compositions, in particular mixtures of fragrance substances or perfume oils, preferably comprise two, three, four, five, six, seven, eight, nine, ten or more fragrance substances. The fragrance mixtures or perfume oils, respectively, are preferably selected from the group of extracts from natural raw materials, such as essential oils, concretes, absolutes, resins, resinoids, balsams, tinctures such as e.g., Ambergris oil; Amyris oil; Angelica seed oil; Angelica root oil; Anise oil; Valerian oil; Basil oil; Tree moss absolute; Bay oil; Mugwort oil; Benzoin resin; Bergamot oil; Beeswax absolute; Birch tar oil; Bitter almond oil; Savory oil; Bucco leaf oil; Cabreuva oil; Cade oil; Calmus oil; Camphor oil; Cananga oil; Cardamom oil; Cascarilla oil; Cassia oil; Cassie absolute; Castoreum absolute; Cedar leaf oil; Cedarwood oil; Cistus oil; Citronella oil; Citron oil; Copaiva balsam; Copaiva balsam oil; Coriander oil; Costus root oil; Cumin oil; Cypress oil; Davana oil; Dill herb oil; Dill seed oil; Eau de brouts absolute; Oak moss absolute; Elemi oil; Tarragon oil; Eucalyptus citriodora oil; Eucalyptus oil; Fennel oil; Spruce needle oil; Galbanum oil; Galbanum resin; Geranium oil; Grapefruit oil; Guaiac wood oil; Gurjun balsam; Gurjun balsam oil, Helichrysum absolute; Helichrysum oil; Ginger oil; Iris root absolute; Iris root oil; Jasmine absolute; Calamus oil; Chamomile oil blue; Chamomile oil Roman; Carrot seed oil; Cascarilla oil; Pine needle oil; Curly mint oil; Caraway seed oil; Labdanum oil; Labdanum absolute; Labdanum resin; Lavandin absolute; Lavandin oil; Lavender absolute; Lavender oil; Lemongrass oil; Lovage oil; Lime oil distilled; Lime oil pressed; Linal oil; Litsea cubeba oil; Bay leaf oil; Macis oil; Marjoram oil; Mandarin oil; Masso bark oil; Mimosa absolute; Musk grain oil; Musk tincture; Muscat sage oil; Muscat oil; Myrrh absolute; Myrrh oil; Myrtle oil; Clove leaf oil; Clove flower oil; Neroli oil; Olibanum absolute; Olibanum oil; Opopanax oil; Orange flower absolute; Orange oil; Origanum oil; Palmarosa oil; Patchouli oil; Perilla oil; Perubalsam oil; Parsley leaf oil; Parsley seed oil; Petitgrain oil; Peppermint oil; Pepper oil; Pimento oil; Pine oil; Poley oil; Rose absolute; Rosewood oil; Rose oil; Rosemary oil; Sage oil Dalmatian; Sage oil Spanish; Sandalwood oil; Celery seed oil; Spicy lavender oil; Star anise oil; Styrax oil; Tagetes oil; Fir needle oil; Tea tree oil; Turpentine oil; Thyme oil; Tolu balsam; Tonka absolute; Tuberose absolute; Vanilla extract; Violet leaf absolute; Verbena oil; Vetiver oil; Juniper berry oil; Wine yeast oil; Wormwood oil; Wintergreen oil; Ylang oil; Hyssop oil; Civet absolute; Cinnamon leaf oil; Cinnamon bark oil; and fractions thereof or ingredients isolated therefrom.

In a further variant of the invention, aroma substances can also be encapsulated as a core material in the form of a single aroma, wherein the core material comprises at least one single aroma substance or mixtures thereof as active ingredient.

However, preferably a single fragrant substance or odorous substance (i.e., chemical compounds having a smell or odour and thus all natural and synthetic substances that impart an olfactorily perceptible odour) or alternatively a mixture of fragrant substances or odorous substances (so-called perfume oil) is used as the active ingredient enclosed by the microcapsule shell. More preferably, said substances and mixtures of substances are imparting a pleasant odour to a consumer product.

In a preferred variant of the present invention, all microcapsules according to the invention are loaded with the same active ingredient(s), for example all of the microcapsules are comprising a fragrance substance and even more preferred the same fragrance substance.

According to an alternative embodiment, the microcapsules according to the invention are encapsulating different classes of active ingredients or different substances among the same class of active ingredients.

Thereby, preferably the core material is selected irrespective of the shell material.

The inventive compositions comprising the microcapsules encapsulating the active ingredient(s) are efficiently incorporated into a variety of solid consumer product formulations allowing for an efficient and targeted release of the active ingredient(s). Thereby the presence of at least three layers, preferably at least three distinct layers, acts as kind of a shock absorbing layer protecting the microcapsules from damage due to mechanical, chemical and/or thermal influences and thus premature release and degradation of the active ingredient(s) during the preparation process of the consumer product.

Moreover, the microcapsules of the invention may further comprise one or more ingredients which improve the visual aesthetics of the final consumer product such as colourants. In addition, additive and/or active ingredients such as optical brighteners, cleaning agents, laundry actives and the like could be comprised by the microcapsules.

### Process for the Preparation

According to a second aspect the present invention relates to a process for the preparation of microcapsules containing at least one ingredient, preferably comprising at least one fragrance, the microcapsules comprise at least three layers surrounding said ingredient, wherein in a first step (a) at least a first layer is prepared, wherein in a second step (b), at least a second layer is prepared and wherein in a third step (c), at least a third layer is prepared, wherein the second layer is prepared after the first layer is hardened and the third layer is prepared after the second layer is hardened.

Preferably, in the first step (a) the first layer is prepared by the following sub-steps:
(i) providing a non-aqueous phase comprising at least one polyisocyanate compound and at least one ingredient, preferably comprising at least one fragrance, and;
(ii) providing an aqueous phase comprising at least one alcohol compound and optionally at least one stabilizer and/or at least one emulsifier;
(iii) emulsifying or dispersing said non-aqueous phase in said aqueous phase to obtain an oil-in-water emulsion or dispersion;
(iv) hardening to obtain the first layer.

Preferably,
in sub-step (i), the at least one polyisocyanate compound is an aromatic polyisocyanate, more preferably with at least two isocyanate groups;
   and/or
in sub-step (ii), the aqueous phase is prepared by solvating at least one alcohol compound comprising at least two hydroxy groups, preferably by solvating a polyol, particularly aromatic polyol and an emulsifier in water.

Preferably, in sub-step (iv), the hardening is performed at a temperature range from 20° C. to 40° C. and the hardening time is between 10 minutes and 120 minutes, preferably between 20 minutes and 60 minutes, more preferably between 25 minutes and 45 minutes.

Hence, in a preferred embodiment, the first layer of the microcapsules is a polyurethane layer.

In sub-step (i), the amount of the at least one polyisocyanate compound is 0.1 wt. % to 0.8 wt. %, preferably 0.2 wt. % to 0.5 wt. %, more preferably 0.25 wt. % to 0.35 wt. %, most preferably 0.27 wt. % to 0.3 wt. %.

In sub-step (i), the amount of the at least one ingredient, preferably comprising at least one fragrance is 10 wt. % to 60 wt. %, preferably 20 wt. % to 55 wt. %, more preferably 35 wt. % to 45 wt. %.

In sub-step (i), the at least one polyisocyanate is a mixture comprising two or more different polyisocyanates, preferably aromatic polyisocyanates each comprising two or more —N═C═O functional groups. Preferably, the amount of aromatic diisocyanate in a mixture comprising said aromatic diisocyanate and an aromatic triisocyanate is 0.2 wt. % to 0.6 wt. %, more preferably 0.25 wt. % to 0.4 wt. % and particularly 0.3 wt. % of said mixture.

In a further embodiment of the invention, the at least one polyisocyanate is a mixture of an aromatic diisocyanate and an aromatic triisocyanate, wherein the aromatic diisocyanate comprises or consist of xylylene diisocyanate, more preferably m-xylylene diisocyanate.

Preferably, in sub-step (i), the at least one polyisocyanate is a mixture of an aromatic diisocyanate and an aromatic triisocyanate, wherein the aromatic diisocyanate comprises or consist of xylylene diisocyanate, more preferably m-xylylene diisocyanate, wherein the amount of the aromatic diisocyanate is 0.2 wt. % to 0.6 wt. %, more preferably 0.25 wt. % to 0.4 wt. % and particularly 0.3 wt. %, and wherein the mixture further comprises at least one lewis base. Preferably, the amount of the least one lewis base is 15 wt. % to 45 wt. %, preferably 20 wt. % to 30 wt. %, particularly 25 wt. % of the mixture of an aromatic diisocyanate and an aromatic triisocyanate, wherein the aromatic diisocyanate comprises or consist of xylylene diisocyanate, more preferably m-xylylene diisocyanate, wherein the amount of the aromatic diisocyanate is 0.2 wt. % to 0.6 wt. %, more preferably 0.25 wt. % to 0.4 wt. % and particularly 0.3 wt. %, wherein the mixture further comprises at least one lewis base.

In a preferred embodiment, the at least one lewis base is ethyl acetate.

In sub-step (ii), the amount of the at least one alcohol compound is 0.01 wt. % to 0.3 wt. %, preferably 0.05 wt. % to 0.2 wt. %, more preferably 0.08 wt. % to 0.12 wt. %.

In sub-step (ii), the amount of the optionally at least one stabilizer and/or at least one emulsifier compound is 1.0 wt. % to 10 wt. %, preferably 2 wt. % to 5 wt. %, more preferably 3 wt. % to 4 wt. %.

### Preferably, in the Second Step (b) the Second Layer Is Prepared by the Following sub-steps:

(v) solvating a melamine-formaldehyde containing precondensate in water or a mixture of melamine and formaldehyde in water to obtain a solution;
(vi) optionally adding at least one catalyst;
(vii) hardening to obtain the second layer.

Preferably, the solution obtained in sub-step (v) is acidified by the at least one catalyst in sub-step (vi) to a pH ranging from 3 to 4, preferably from 3.25 to 3.75, more preferably to 3.5.

Optionally, in sub-step (vii), the hardening is performed at a temperature range from 30° C. to 70° C., preferably from 40° C. to 60° C., more preferably from 45° C. to 55° C.

In sub-step (vi), the amount of the at least one catalyst is 0.05 wt. % to 0.5 wt. %, preferably 0.1 wt. % to 0.4 wt. %, more preferably 0.2 wt. % to 0.3 wt. %.

the solution obtained in sub-step (v) is acidified by the at least one catalyst in sub-step (vi) to a pH ranging from 3 to 4, preferably from 3.25 to 3.75, more preferably to 3.5.

Hence, in a preferred embodiment, the second layer of the microcapsules is a melamine-formaldehyde layer.

In sub-step (v), the amount of the melamine-formaldehyde containing precondensate is 0.5 wt. % to 5 wt. %, preferably 1 wt. % to 3.5 wt. %, more preferably 1.5 wt. % to 2 wt. %.

Preferably, in the third step (c) the third layer is prepared by the following sub-steps:
(viii) adding at least one alcohol compound to the second layer;
(ix) hardening to obtain the third layer.

In an embodiment of the invention,
in sub-step (viii), the at least one alcohol compound is preferably an aromatic polyol, more preferably Phloroglucinol and/or Resorcinol;
   and/or
in sub-step (viii), the at least one alcohol compound is added as a solid, preferably in portions.

In sub-step (viii), the amount of the the at least one alcohol compound is 0.01 wt. % to 0.3 wt. %, preferably 0.05 wt. % to 0.2 wt. %, more preferably 0.08 wt. % to 0.12 wt. %.

In another embodiment of the invention, in sub-step (ix), hardening is performed at a temperature range from 20° C. to 100° C., preferably from 70° C. to 90° C., more preferably from 75° C. to 85° C. and the hardening time is between 30 minutes and 360 minutes, preferably between 60 minutes and 240 minutes.

Hence, in a preferred embodiment, the third layer of the microcapsules is a preferably a phenolic resin layer.

Optionally, in a further sub-step (x), at least one amino-containing compound, preferably urea, is added, preferably as a solid after sub-step (ix).

In sub-step (x), the amount of the at least one amino-containing compound, preferably urea is 0.1 wt. % to 0.8 wt. %, preferably 0.25 wt. % to 0.6 wt. %, more preferably 0.4 wt. % to 0.5 wt. %.

Optionally, in a further sub-step (xi), cooling to a temperature from 40° C. to 50° C., preferably from 42° C. to 48° C., particularly to 45° C. is performed after sub-step (ix).

Optionally, in a further sub-step (xii), the pH value is adjusted to a range from 5 to 6, preferably to 5.3 to 5.7, more preferably to 5.5 after sub-step (xi) to obtain a slurry.

Optionally, in a further sub-step (xiii) the slurry obtained from sub-step (xii) is filtered and thickened.

In a preferred embodiment of the invention, in further sub-step (x), at least one amino-containing compound, preferably urea, is added, preferably as a solid after sub-step (ix). In a subsequent sub-step (xi), cooling to a temperature from 40° C. to 50° C., preferably from 42° C. to 48° C., particularly to 45° C. is performed followed by pH value adjustment to a range from 5 to 6, preferably to 5.3 to 5.7, more preferably to 5.5 after in a sub-step (xii) to obtain a slurry. Finally, in a further sub-step (xiii) the slurry obtained from sub-step (xii) is filtered and thickened. The microcapsules thus obtained three separate layers and hence have improved stabilities beside excellent release properties.

In a preferred embodiment, the at least first layer and the at least second layer and the at least third layer are prepared in a single pot. Particularly, hardening steps are performed after each layer-formation. This allows an efficient and simplified preparation of microcapsules with excellent release properties and improved stabilities.

In order to achieve a homogeneous distribution of microcapsules it is advantageous to use microcapsules having a particle size ranging from 150 μm to 5 mm, preferably in the rage from 500 μm to 3 mm. Thus, according to a preferred variant of the present invention the particle size of the microcapsules according to present invention is preferably in the range from 150 μm to 5 mm, preferably in the rage from 500 μm to 3 mm.

Additionally, in a further aspect of the present invention relates to microcapsules prepared according to the inventive process.

Additionally, in a further aspect of the present invention relates to microcapsules prepared according to the inventive process.

### Consumer Products

In another aspect, the present invention relates to the use of the microcapsules according to the invention for the preparation of consumer product formulations. The terms “consumer product formulation” and “consumer product” are used synonymously within the context of the present invention.

Particular advantages of the inventive microcapsules obtained by the process for preparation according to the invention were found in view of the preparation of solid consumer products such as powder detergents, laundry soaps, solid fabric softeners, deo sticks and the like. Thus, preferably the consumer product according to the present invention is a solid consumer product.

Therefore, in another preferred variant the present invention also relates to the use of the microcapsules according to the present invention for the preparation of consumer products and formulations thereof, wherein said consumer product or consumer product formulation is a solid product such as a textile care product (such as a powder detergent, laundry soap, solid fabric softener), a solid soap, a household product (such as a solid WC-cleaner, solid all-purpose cleaners, powdered carpet cleaners, powdery washing agents for washing dishes or for cleaning various surfaces), a personal care product (such as a deodorant, soap), scent boosters, fragrance enhancers, a pharmaceutical product, and mixtures thereof.

Finally, in another aspect the present invention relates to the consumer product or consumer product formulation as such comprising or consisting of the microcapsules according to the invention.

The inventive microcapsules allow for the efficient incorporation of active ingredients into solid product formulations allowing for an efficient and targeted release of said active ingredients.

### EXAMPLES

Hereinafter, the present invention is described in more detail and specifically with reference to the examples, which however are not intended to limit the present invention.

### Example 1: Preparation of Microcapsules

According to a first example Lupasol PA-140 was dissolved in water and tannic acid was added to the solution. A fragrance oil and Takenate D-110 N were mixed and added. To obtain the first layer, hardening was performed at 35° C. for 30 minutes. Next, a solution of a melamine-formaldehyde precondensate in water was added, followed by addition of a catalyst to start the polymerisation reaction. As a catalyst, formic acid was added and the pH was adjusted to 3.5. To obtain the second layer, hardening was performed by heating to 50° C. Next, Phloroglucinol was added and the temperature was increased to perform a further hardening step to obtain the third layer. Microcapsules were obtained after addition of Urea, cooling to 45° C., adjusting the pH to 5.5 followed by thickening.

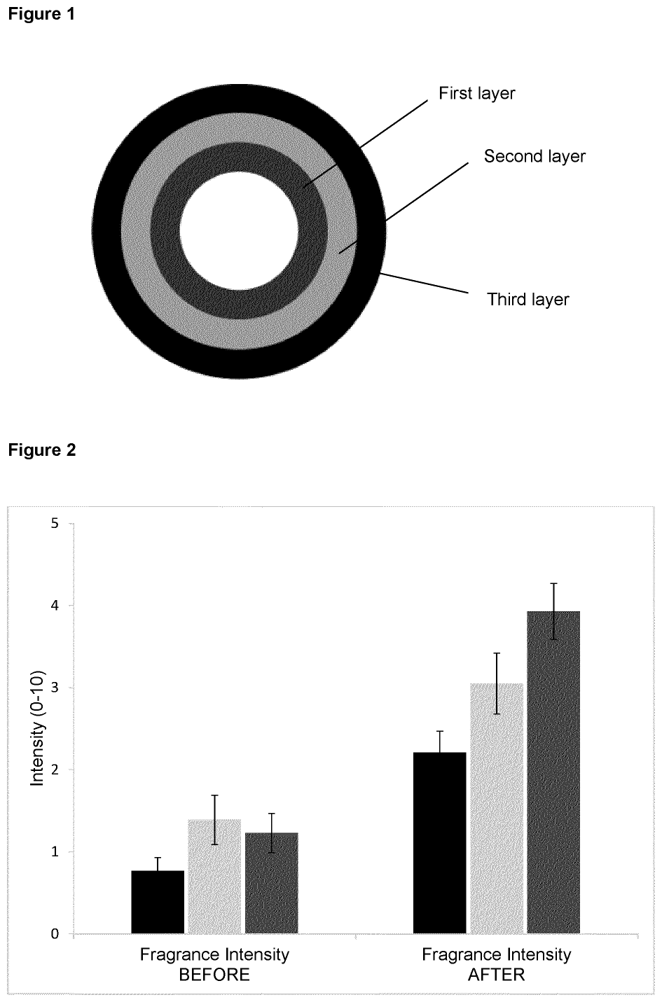

Hence, microcapsules according to the invention comprise at least one ingredient, preferably comprising at least one fragrance, and at least three different layers surrounding said ingredient, wherein at least one layer is a polyurethane layer and at least one layer is one of a melamine-formaldehyde layer or a phenolic resin layer (see FIG. 1).

Specifically, microcapsules obtained by the invention as described above could comprise a first layer being a polyurethane layer. The first layer could also be understood as an inner layer. Accordingly, the third layer, presently a phenolic resin or formaldehyde-phenol-resin or bakelite respectively could be understood as an outer layer. The second layer, presently a melamine-formaldehyde layer is thus arranged between the first and the third layer.

According to the invention, the microcapsules may contain more layers. Preferably, said layers could be but are not limited to polyurethanes, phenolic resins and/or melamine-formaldehydes.

The sensory evaluation was performed as follows: once the microcapsules according to the present invention were prepared as described above, they were combined with commercially available powder detergent.

### Microcapsules Application in Powder Detergent

Microcapsules were sprayed (45 Psi, 5.5 kg/hour) and collected, simulating a praying process application. Sprayed microcapsules are used to apply in powder detergent non fragrance base (0.11%) and mixed to have a homogenized application.

### Protocol for Hand Washing Towels with Powder Detergent

Per towel, 5 g of powder detergent were diluted in 1 L of tap water in a container. Towels were placed in the container and moved 10 times in circles in order to simulate washing movements. Towels were then for 15 minutes kept in the container, squeezed or scrubbed by hands and rinsed with 1 L of water (each towel). Towels were then squeezed again and air-dried.

### Preparation of Samples

Dry towels were evaluated in fragrance intensity each, in pre and post rubbing. Therefore, a quantitate descriptive test or profiling test was performed which is a descriptive test used to determine the intensity of several attributes in a product. An evaluation Panel trained testing the towels using following evaluation scale from 1 to 10.

8 to 10 test persons assessed the fragrance intensity on a scale of 1 (non-perceptible) to 10 (extremely strong).

The results of the sensory evaluation are shown in FIG. 2. As shown, microcapsules according to the invention have improved release properties (post rubbing).

1. Microcapsules, comprising at least one ingredient, preferably comprising at least one fragrance, and at least three different layers surrounding said ingredient, wherein at least one layer is a polyurethane layer and at least one layer is one of a melamine-formaldehyde layer or a phenolic resin layer.

2. Microcapsules according to claim 1, said microcapsules comprise at least one polyurethane layer and at least one melamine-formaldehyde layer and at least one phenolic resin layer.

3. Microcapsules according to claim 2, wherein the at least one melamine-formaldehyde layer is arranged between the at least one polyurea layer and the at least one phenolic resin layer.

4. Microcapsules according to claim 1, wherein the at least one phenolic resin layer is an outer layer.

5. Microcapsules according to claim 1,
wherein the at least one polyurethane layer is formed by tannic acid and at least one polyisocyanate, preferably at least one aromatic polyisocyanate, more preferably the at least one polyisocyanate is a mixture of an aromatic diisocyanate and an aromatic triisocyanate,
wherein the at least one melamine-formaldehyde layer is formed by at least one melamine-formaldehyde pre-condensate in water or by at least melamine and formaldehyde as single compounds and the at least one phenolic resin layer is formed by at least one aromatic polyol, preferably by Phloroglucinol and/or Resorcinol.

6. Microcapsules according to claim 1, wherein the at least three different layers are separated.

7. Process for the preparation of microcapsules containing at least one ingredient, preferably comprising at least one fragrance, the microcapsules comprise at least three layers surrounding said ingredient, wherein in a first step (a) at least a first layer is prepared, wherein in a second step (b), at least a second layer is prepared and wherein in a third step (c), at least a third layer is prepared, wherein the second layer is prepared after the first layer is hardened and the third layer is prepared after the second layer is hardened.

8. Process according to claim 7, wherein in the first step (a) the first layer is prepared by the following sub-steps:
(i) providing a non-aqueous phase comprising at least one polyisocyanate compound and at least one ingredient, preferably comprising at least one fragrance, and;
(ii) providing an aqueous phase comprising at least one alcohol compound and optionally at least one stabilizer and/or at least one emulsifier;
(iii) emulsifying or dispersing said non-aqueous phase in said aqueous phase to obtain an oil-in-water emulsion or dispersion;
(iv) hardening to obtain the first layer.

9. Process according to claim 8, wherein one or more of the following applies:
in sub-step (i), the at least one polyisocyanate compound is an aromatic polyisocyanate, more preferably with at least two isocyanate groups, even more preferably the at least one polyisocyanate is a mixture of an aromatic diisocyanate and an aromatic triisocyanate;
in sub-step (ii), the aqueous phase is prepared by solvating at least one alcohol compound comprising at least two hydroxy groups, preferably by solvating a polyol, particularly aromatic polyol and an emulsifier in water;
in sub-step (iv), the hardening is performed at a temperature range from 20° C. to 40 ° C. and the hardening time is between 10 minutes and 120 minutes, preferably between 20 minutes and 60 minutes, more preferably between 25 minutes and 45 minutes

10. (canceled)

11. Process according to claim 7, wherein in the second step (b) the second layer is prepared by the following sub-steps:
(v) solvating a melamine-formaldehyde containing pre-condensate in water or a mixture of melamine and formaldehyde in water to obtain a solution;
(vi) optionally adding at least one catalyst;
(vii) hardening to obtain the second layer.

12. Process according to claim 11, wherein one or more of the following applies:
the solution obtained in sub-step (v) is acidified by the at least one catalyst in sub-step (vi) to a pH ranging from 3 to 4, preferably from 3.25 to 3.75, more preferably to 3.5;
in sub-step (vii), the hardening is performed at a temperature range from 30° C. to 70° C., preferably from 40° C. to 60° C. more preferably from 45° C. to 55° C.

13. (canceled)

14. Process according to claim 7, wherein in the third step (c) the third layer is prepared by the following sub-steps:
(viii) adding at least one alcohol compound to the second layer;
(ix) hardening to obtain the third layer.

15. Process according to claim 14, wherein
in sub-step (viii), the at least one alcohol compound is preferably an aromatic polyol, more preferably Phloroglucinol and/or Resorcinol;
and/or
in sub-step (viii), the at least one alcohol compound is added as a solid, preferably in portions.

16. Process according to claim 14, wherein in sub-step (ix), the hardening is performed at a temperature range from 20° C. to 100° C., preferably from 70° C. to 90° C., more preferably from 75° C. to 85° C. and the hardening time is between 30 minutes and 360 minutes, preferably between 60 minutes and 240 minutes.

17. Process according to claim 14, wherein in a further sub-step (x), at least one amino-containing compound, preferably urea, is added, preferably as a solid after sub-step (ix).

18. Process according to claim 14, wherein in a further sub-step (xi), cooling to a temperature from 40° C. to 50° C. preferably from 42° C. to 48° C., particularly to 45° C. is performed after sub-step (ix).

19. Process according to claim 18, wherein in a further sub-step (xii), the pH value is adjusted to a range from 5 to 6, preferably to 5.3 to 5.7, more preferably to 5.5 after sub-step (xi) to obtain a slurry.

20. Process according to claim 19, wherein in a further sub-step (xiii) the slurry obtained from sub-step (xii) is filtered and thickened.

21. Process according to claim 7, wherein the at least first layer and the at least second layer and the at least third layer are prepared in a single pot.

22. (canceled)

23. A consumer product selected from the group consisting of detergents and cleaning agents, personal care products, nutritional or recreational preparations, cosmetic or pharmaceutical preparations, perfumed, perfumed or products to be perfumed, preferably fragrance rinsers, laundry perfumes, the consumer product comprising at least one microcapsule according to claim 1.

24. (canceled)
